(19) United States
(12) Patent Application Publication
(10) Pub. No.: US 2004/0137431 A1
(43) Pub. Date: Jul. 15, 2004

(54) Target sequences for the detection of the west nile virus

(76) Inventors: Ramon Eritja, Barcelona (ES); Martin Munzer, Parkland, FL (US); Martin J. Lopez, Vancouver (CA)

Correspondence Address: DUANE A. STEWART III. ESQ., BUCHANAN INGERSOLL PC, ONE OXFORD CENTRE, 20th FLOOR., 301 GRANT STREET, PITTSBURGH, PA, 15219, US

(21) Appl. No.: 10/700,118

(22) Filed: Nov. 3, 2003

Related U.S. Application Data
(60) Provisional application No. 60/423,508, filed on Nov. 4, 2002.

(51) Int. Cl. C12Q 1/70; C12Q 1/68

(52) U.S. Cl. 435/5

(57) ABSTRACT

The invention provides a method of capturing the ribo-nucleic acid (RNA) specific to the West Nile Virus (WNV); specifically it relates to the target sequences within the WNV genome that can be used to capture the specific RNA material for identification and/or provide target regions for the use of specific probes that inhibit the reproduction of the WNV in vivo, in the form of a gene therapy drug.

This utility patent application claims the benefit of co-pending U.S. Provisional Patent Application Serial No. 60/423,508, filed Nov. 4, 2002, entitled “Target Sequences for the Detection of the West Nile Virus,” having named applicants as inventors, namely Martin J. Lopez, Ramon Eritja, and Martin Munzer. The entire contents of U.S. Provisional Patent Application Serial No. 60/423,508 are incorporated by reference into this utility application.

### COMPUTER READABLE FORM

This application contains a sequence listing in both written and computer readable form. The information recorded in computer readable form is identical to the written (on paper) sequence listing.

### BACKGROUND OF THE INVENTION

1. Field of the Invention

This invention relates to the specific target sites available for the capture or inhibition of RNA related to the West Nile Virus (WNV). The recent outbreak and rapid spread of the WNV in the United States has made it necessary for the development of diagnostic assays for the identification of the virus in patients (defined as any member of the animal kingdom including humans), environmental samples such as the testing of water samples for the presence of the virus in mosquitoes or mosquito larvae, and most importantly for the testing of donated blood, tissue or organs. It has recently been determined that the virus, which normally only severely affects patients who possess a compromised immune system, can be transferred by donated blood, organs and tissues from otherwise healthy or non-symptomatic donors who unknowingly carry the virus. The recipients are generally in an immune compromised state due to the conditions that lead them to require the donated product, thus making them highly susceptible to the infection, which can lead to severe complications and death.

2. Description of the Related Art

Methods for detecting target nucleic acids are set forth in U.S. Pat. Nos. 5,962,225 and 6,100,040, both issued to Elliot R. Ramberg. The disclosures of those patents are incorporated by reference herein.

Parallel-stranded hairpins are discussed in the following papers, all of which are incorporated by reference herein:

Aviñó et al., 2002 Nucleic Acids Res., 30: 2609;

E. Cubero et al., 2002 J. Am. Chem. Soc., 124:3133;

Cubero et al., 2001 Nucleic Acid Res., 29: 2522; and

R. Güimil Garcia, et al. 1999. Nucleic Acid Res., 27:1991.

### SUMMARY OF THE INVENTION

The invention provides specific regions that can be targeted with parallel-stranded hairpin probes (PSH probes) or other nucleic acid probes that can be used for the capture of the RNA specific to the WNV for further analysis and detection.

The invention further provides for the use of these specific RNA sequences as target regions for gene therapy drugs used to treat patients infected with the WNV.

The invention further provides a method for testing a sample for the presence of at least one strain of West Nile Virus comprising providing a sample, the sample optionally containing West Nile Virus RNA, and exposing the sample to an oligomer having a targeting base sequence substantially complementary to at least about seven consecutive bases in a WNV target sequence, then allowing the oligomer to hybridize with the WNV RNA to form a hybrid, then detecting the hybrid, thereby detecting the presence of at least one strain of the West Nile Virus. Another embodiment includes the method of testing a sample for the presence for a strain of WNV as described above, where the WNV target sequence includes any of the sequences set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ and SEQ ID NO: 9.

Another embodiment of the invention includes a nucleic acid probe for detecting a target sequence of West Nile Virus RNA optionally present in a sample, the nucleic acid probe comprising a targeting base sequence that is substantially complementary to at least about seven consecutive bases in a West Nile Virus target sequence.

Another embodiment of the invention includes a method for treating patients having the WNV, comprising administering to that patient a therapeutic amount of a composition capable of binding the RNA of the WNV, wherein the composition comprises an oligonucleotide having a base sequence that is substantially complementary to at least about seven consecutive bases in a West Nile Virus target sequence.

Another embodiment of the invention includes a method for capturing and/or reporting RNA of the WNV, including the steps of providing at least one oligomer probe. The oligomer probe includes a targeting base sequence substantially complementary to at least about seven consecutive bases in a WNV target sequence. The oligomer probe may further include an attached magnetic bead. The method further includes providing a sample, optionally containing RNA of the WNV, and combining the oligomer probe with the sample, forming at least one probe-RNA hybrid, and finally separating the probe-RNA hybrid from the sample by applying the magnetic field to the probe-sample mixture.

In some embodiments of the invention, the oligomer bearing the targeting base sequence may be a hairpin probe. The hairpin probe may optionally contain at least one 8-aminopurine.

### BRIEF DESCRIPTION OF THE FIGURES

FIG. 1 displays the concept of viral RNA target enrichment by way of capturing specific targets by forming a highly stable triplex structure.

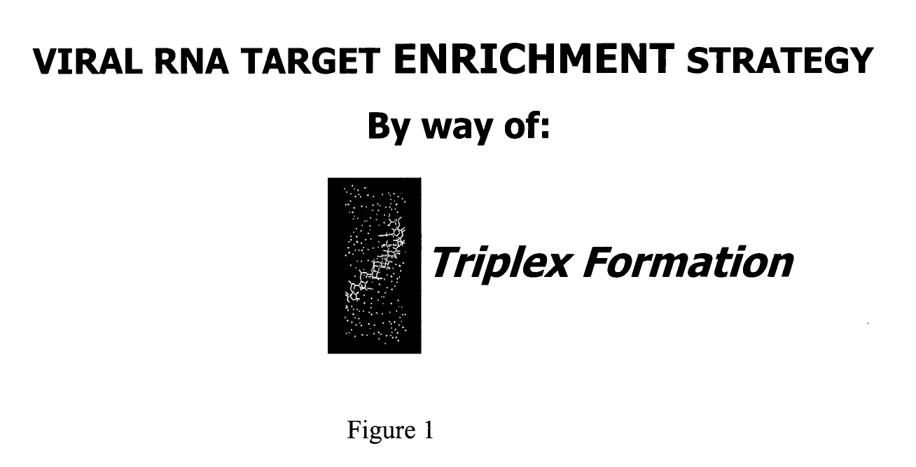

FIG. 2 describes the targeting of the polypyrimidine regions within a genome by using PSH probes containing amino modified substituted bases to form a highly stable triplex structure using the specific region in the target RNA.

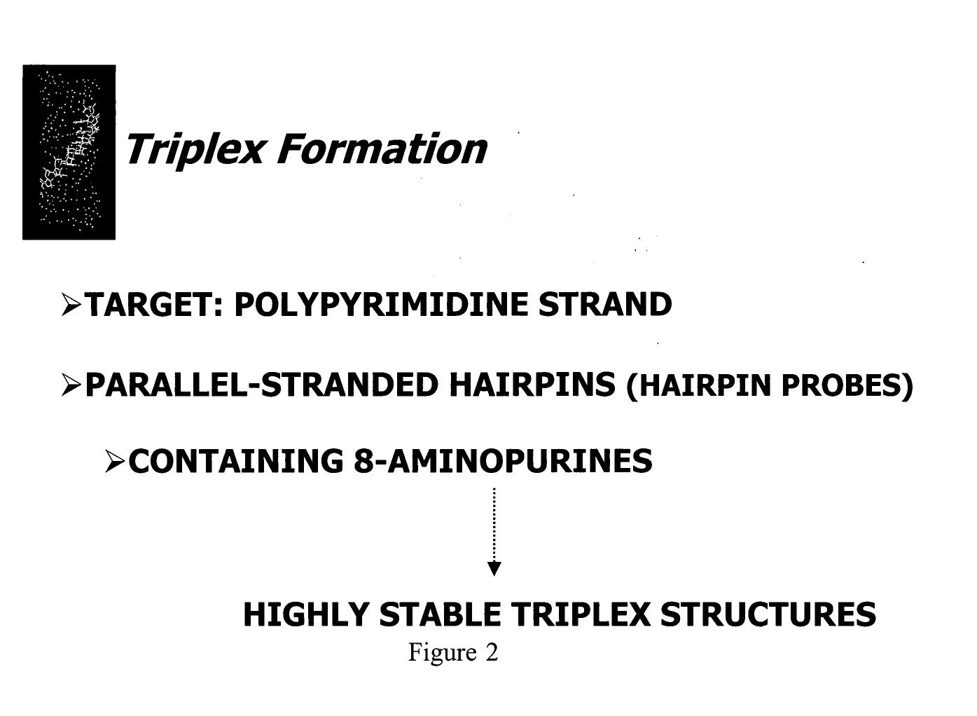

FIG. 3 graphically depicts the resulting triplex structure with a model sequence.

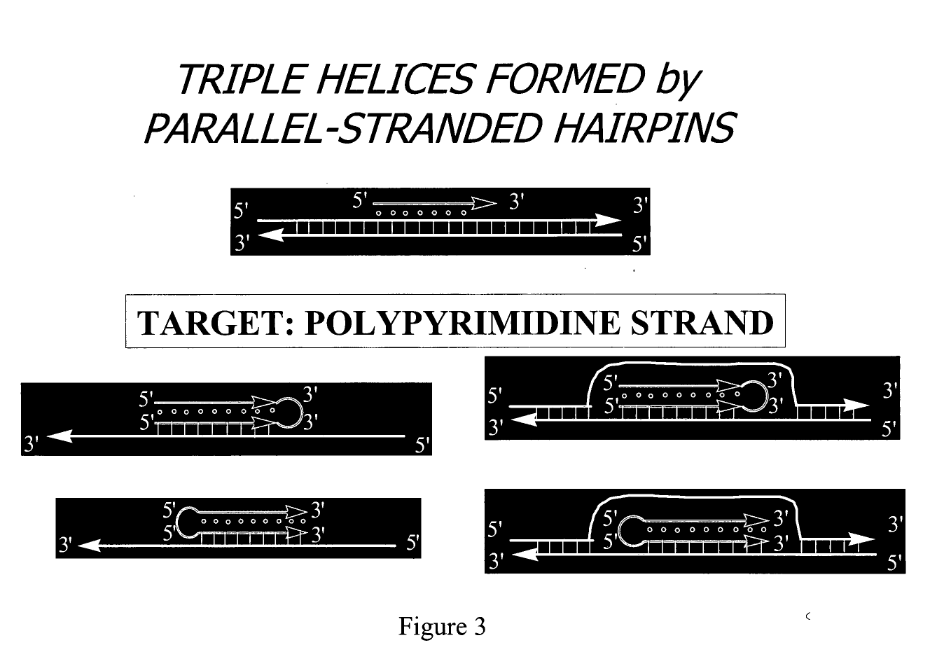

FIG. 4 shows a chemical composition and a method of synthesizing the PSH probes for the present invention.

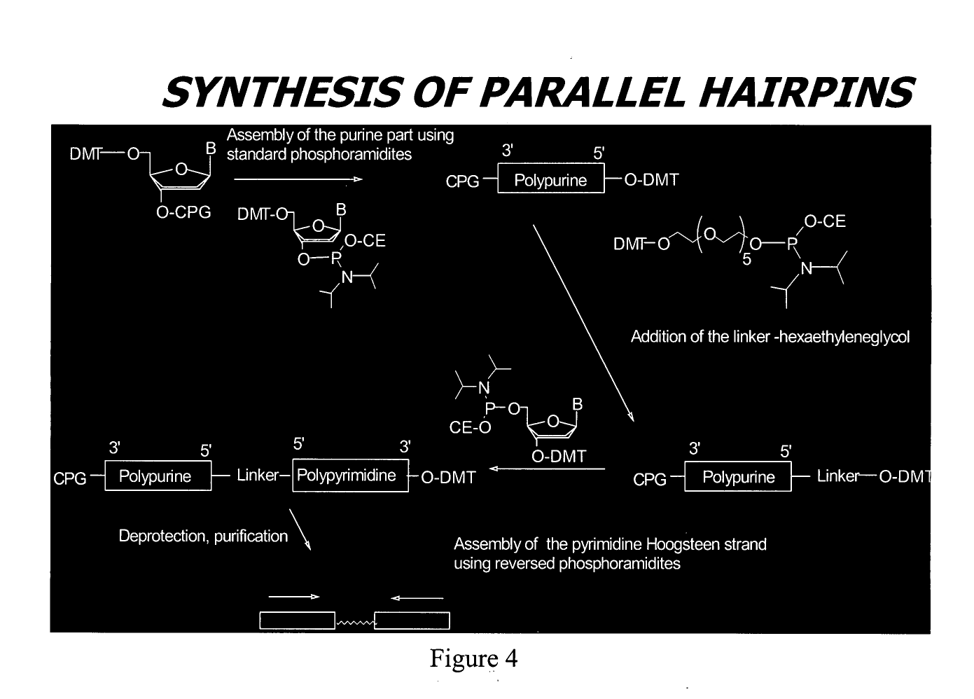

FIG. 5 shows two embodiments of asymmetric branching units that can be used in the synthesis process.

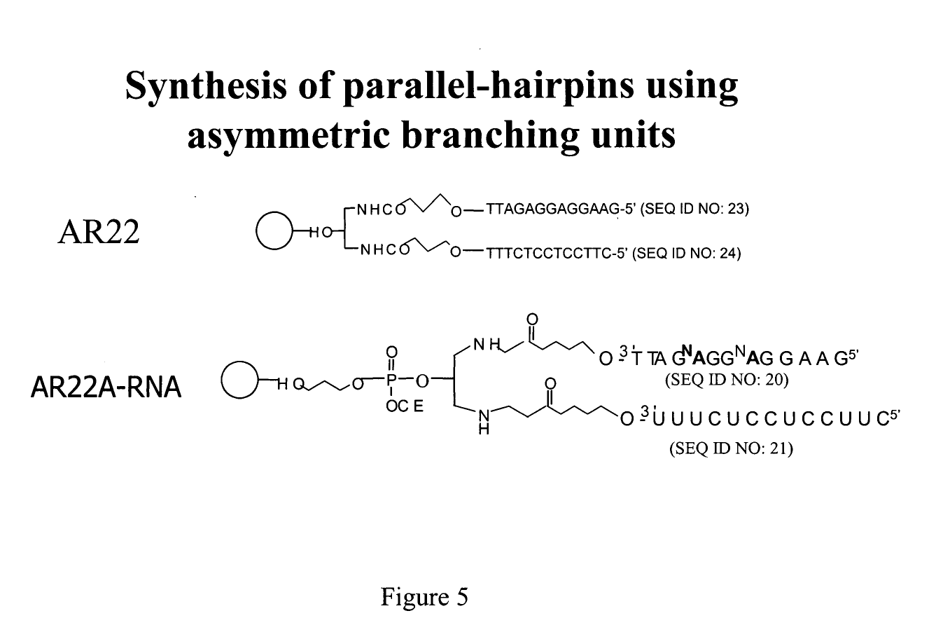

FIG. 6 is representative of a WNV infected sample.

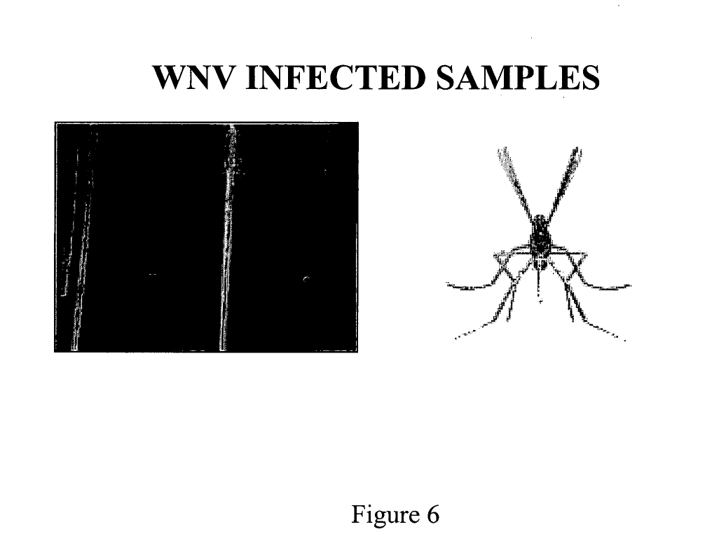

FIG. 7 illustrates steps that may be used for analysis of a WNV infected sample. First the RNA must be extracted from the sample, then a parallel-stranded hairpin probe is introduced to capture the specific targeted WNV, finally a detection system identifies the presence or absence of the WNV.

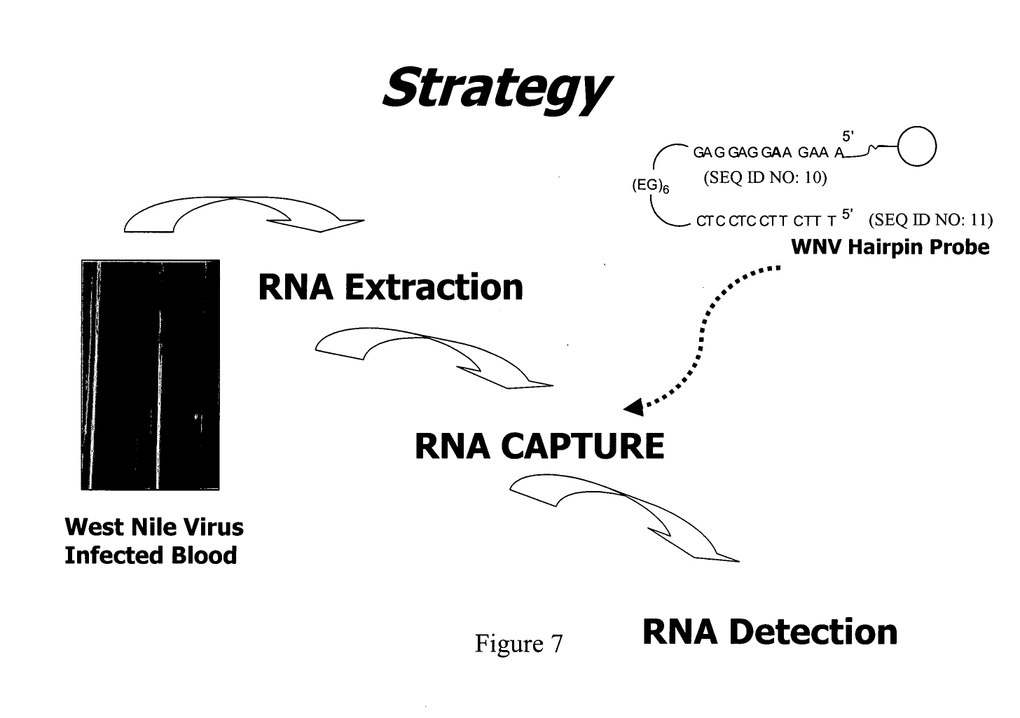

FIG. 8 illustrates how the PSH probes attached to magnetic beads can be used to capture the viral RNA. The PSH probes can be attached to the magnetic beads by way of biotin-streptavidin conjugates or can be directly synthesized to the beads.

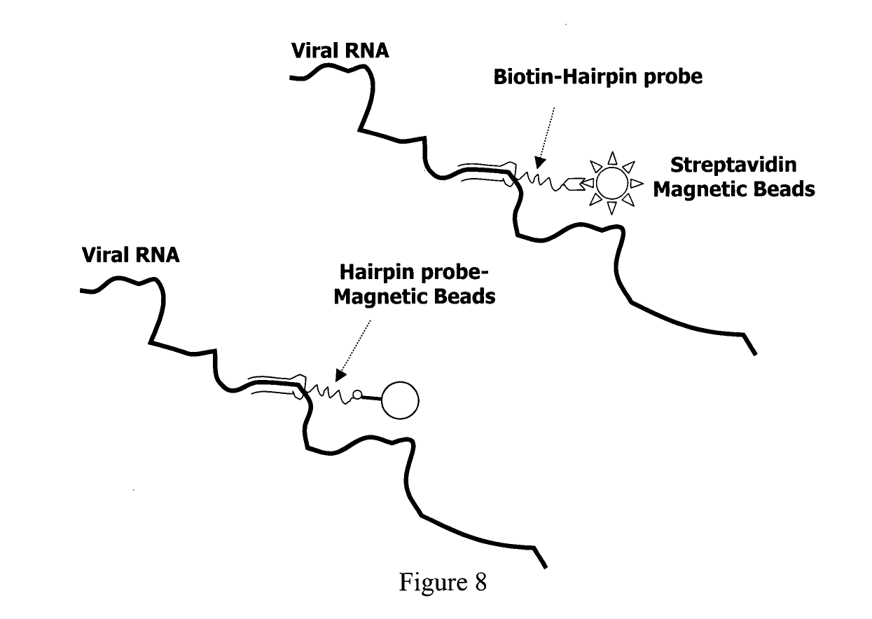

FIG. 9 depicts a robotic analyzer that performs automated magnetic bead separation.

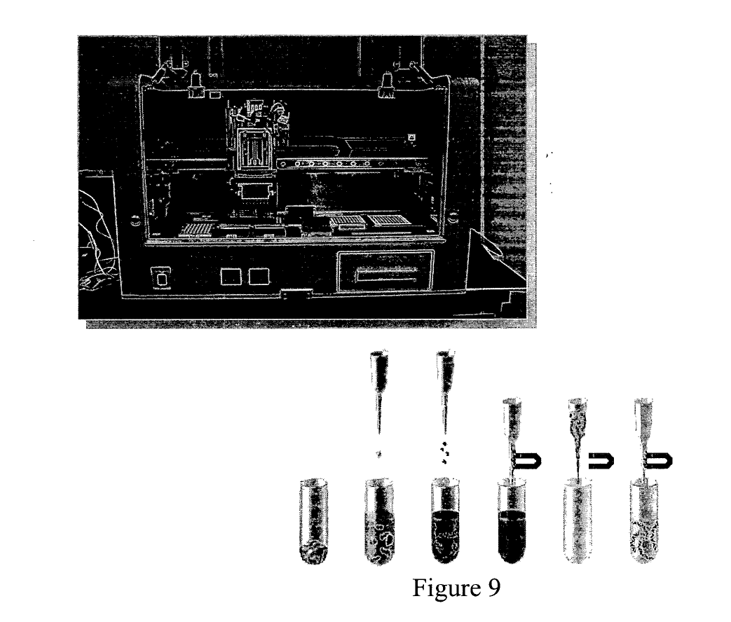

FIG. 10 shows a single nucleotide cross-section of a triplex DNA formation.

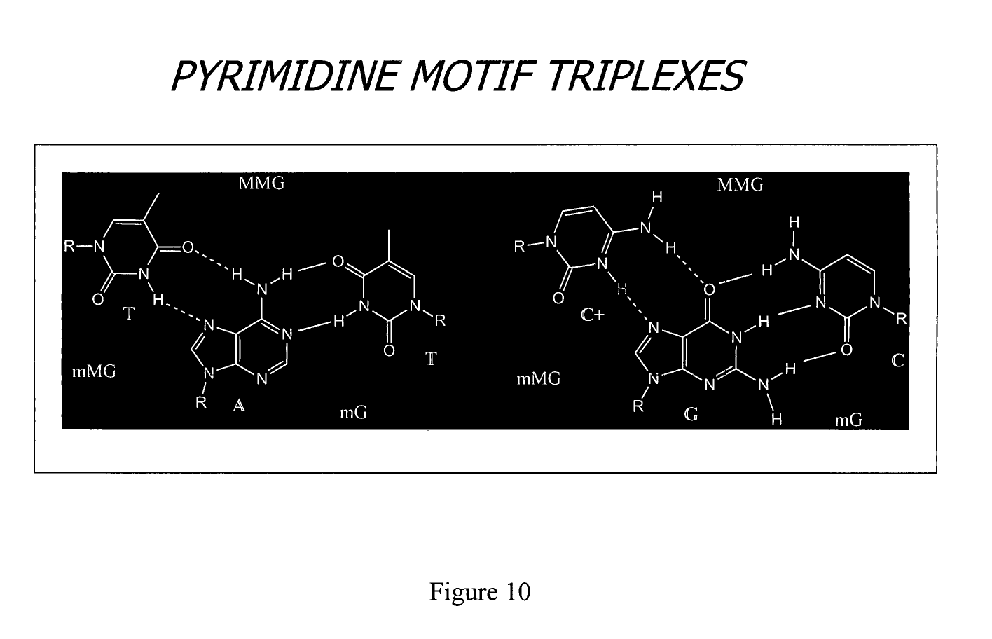

FIG. 11 shows a single nucleotide cross-section of a triplex DNA formation formed with 8-aminopurine base substitutions. Two embodiments of the present invention are illustrated, the 8-aminoadenine and 8-aminoguanine substitutions.

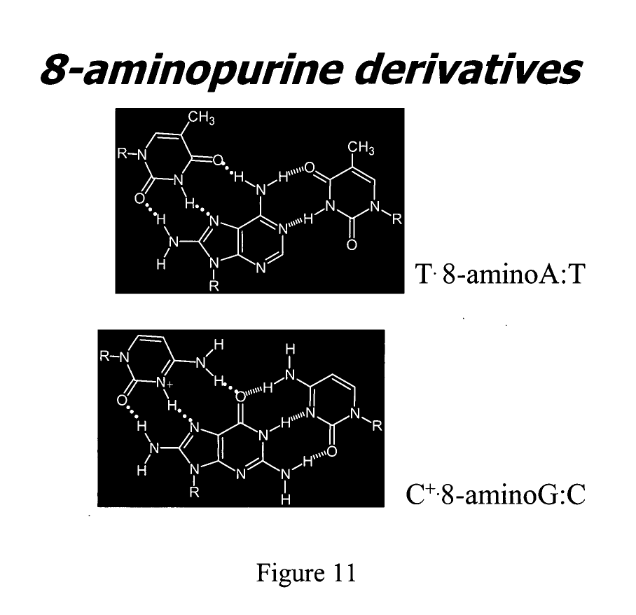

FIG. 12 illustrates the increased structural stability of the 8-amino group substituted bases in the DNA triplex formation by way of the measured binding energy.

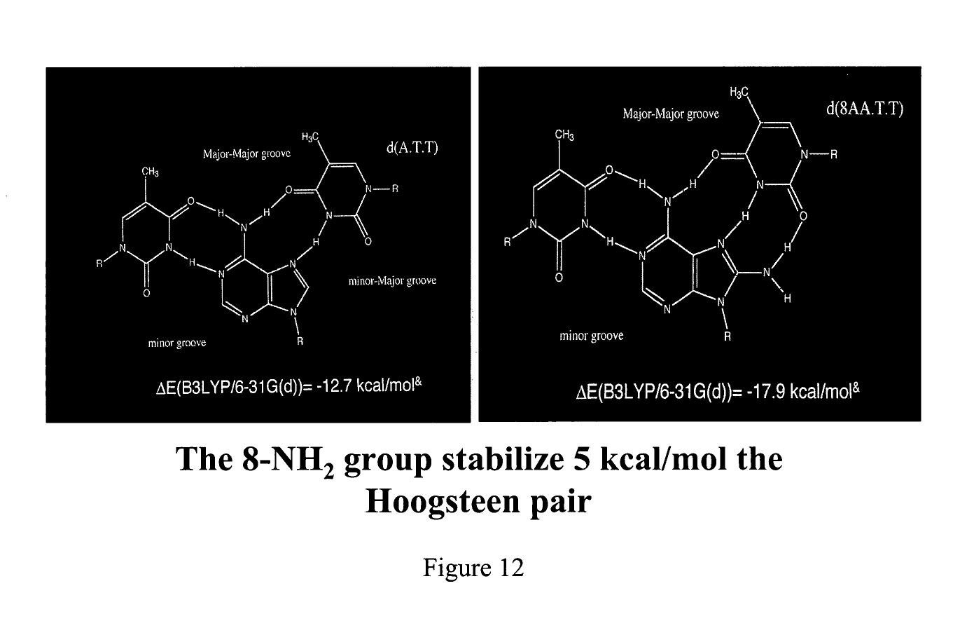

FIG. 13 compares the measured melting temperatures of unmodified PSH probes bound to a single stranded DNA target, with the measured melting temperatures of PSH probes bound to both 8-aminoadenine and 8-aminoguanine of the present invention. PSH probes are joined at the 3 prime ends.

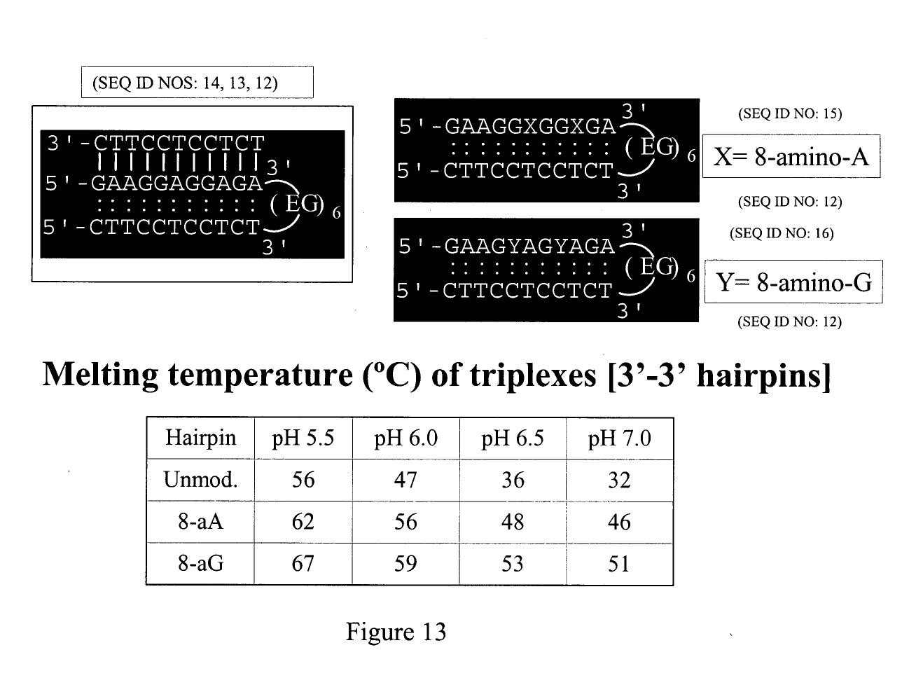

FIG. 14 compares the measured melting temperatures of unmodified PSH probes bound to a single stranded DNA target with PSH probes modified with both 8-aminoadenine and 8-aminoguanine of the present invention; PSH probes are joined at the 5 prime ends.

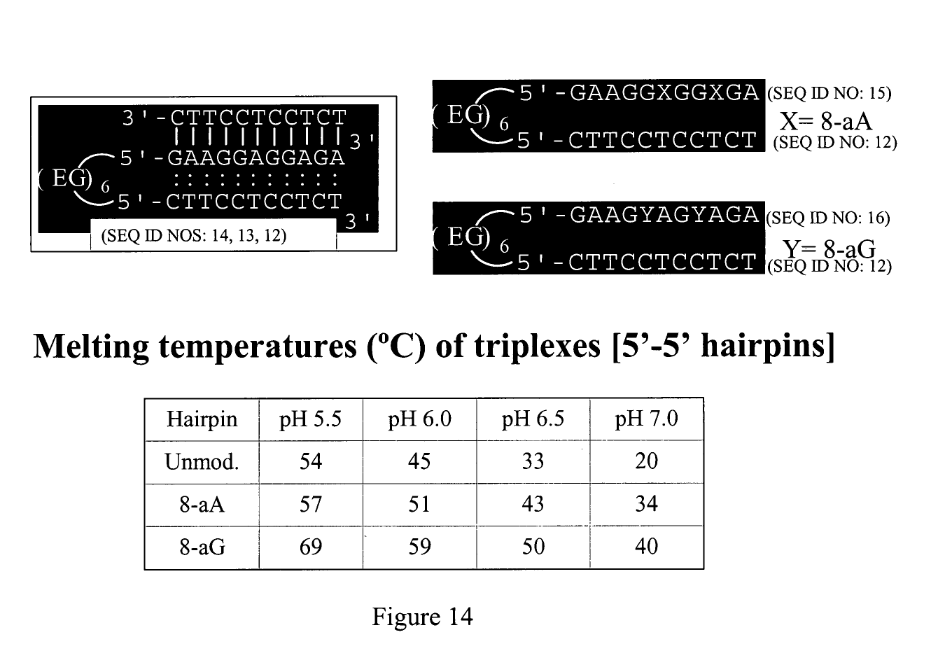

FIG. 15 compares the data from a DNA uninterrupted polypyrimidine track with the data from a polypyrimidine track with one guanine interruption.

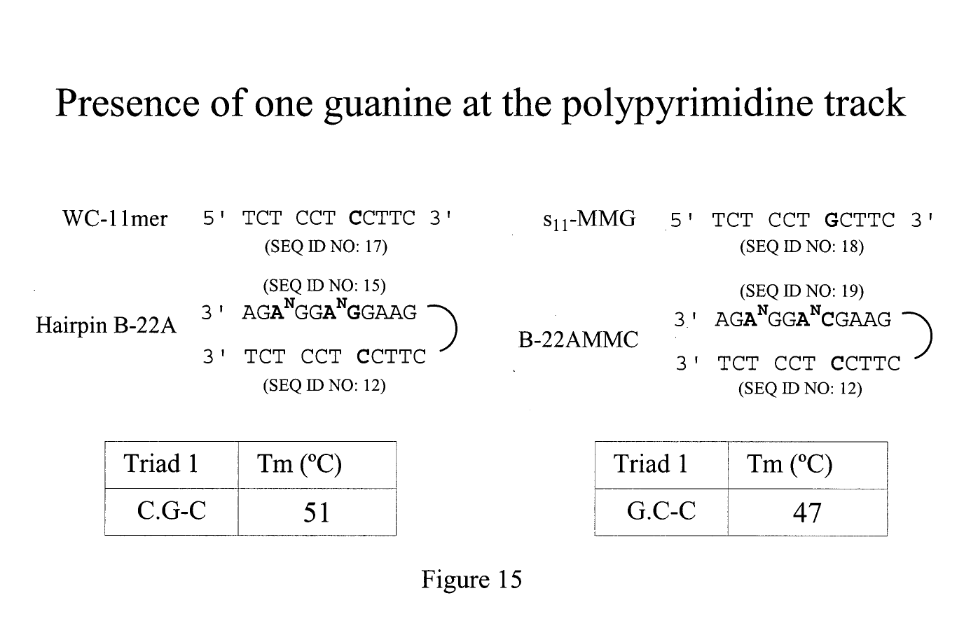

FIG. 16 presents the melting temperature of an amino modified PSH probe with an RNA target.

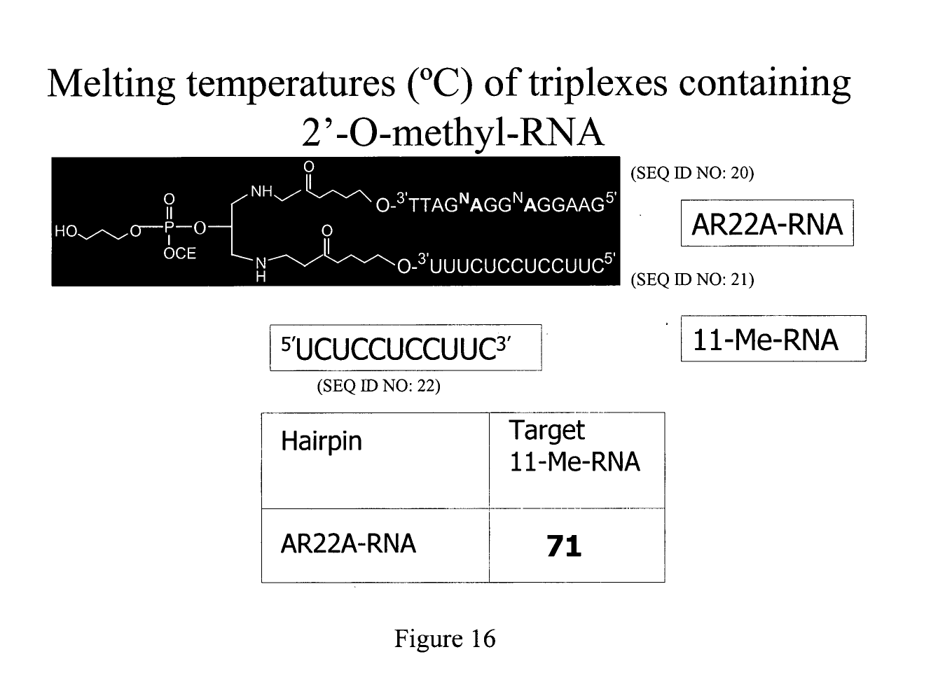

FIG. 17 presents data by way of circular dichroism (CD) and nuclear magnetic resonance (NMR).

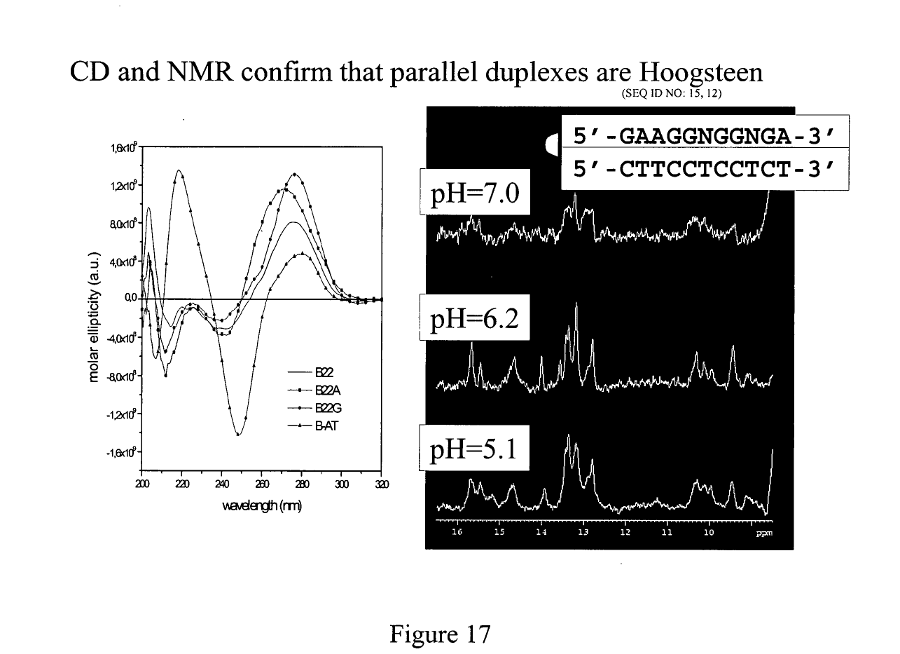

FIG. 18 presents data that indicates the presence of triplex formation with a single stranded DNA target using PSH probes by way of gel-shift and NMR measurements.

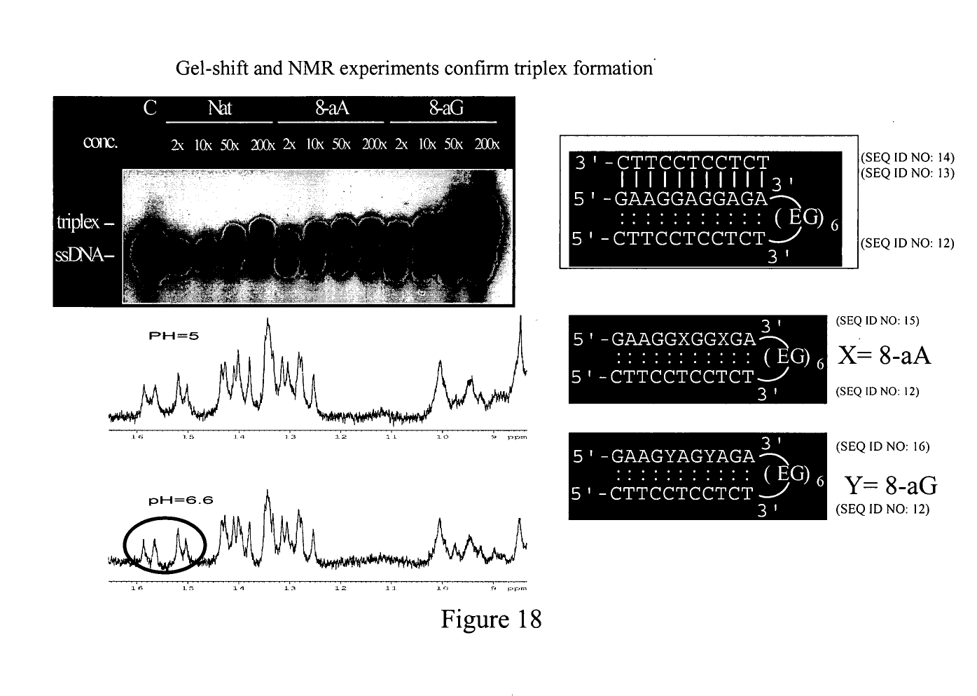

FIG. 19 presents nine candidate sites within the WNV genome that can be used for triplex formation using PSH or other probes.

FIG. 20 demonstrates the effect of triplex formation on the Hoogsteen strand.

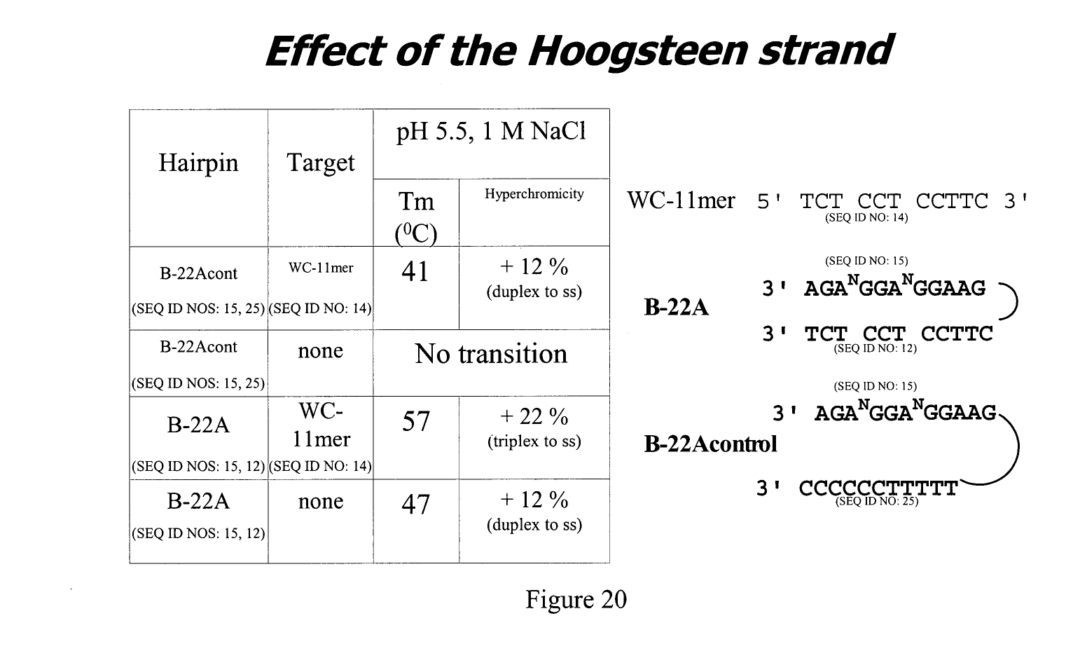

### DETAILED DESCRIPTION OF THE INVENTION

The invention provides specific regions that can be targeted with PSH probes or other nucleic acid probes that can be used for the capture of the RNA specific to the WNV for further analysis and detection. The invention further provides for the use of these specific RNA sequences as target regions for gene therapy drugs used to treat patients infected with the WNV.

West Nile Virus Target Regions

The following sequences are specifically well suited for the capture or inhibition of the WNV by way of PSH probe designs:

| | |
|---|---|
| CTGCTCTCCCTTCTCTT (position 6793) | (SEQ ID NO: 1) |
| CTCTTCCTCTCCGT (position 2443) | (SEQ ID NO: 2) |
| TTCTTCCTCCTCATGC (position 6670) | (SEQ ID NO: 3) |
| CTCCACTTCCTCAAT (position 9727) | (SEQ ID NO: 4) |
| CACTCCTTTTTTGCC (position 7262) | (SEQ ID NO: 5) |
| CTTCCCCTTCGTC (position 7170) | (SEQ ID NO: 6) |
| CCCTTTTGTTTCA (position 2043) | (SEQ ID NO: 7) |
| CTGTTTTCTTTCA (position 3857) (negative blast) | (SEQ ID NO: 8) |
| CCTTTTTCTGTT (position 6740) | (SEQ ID NO: 9) |

The following sequences have homology with 25-30 different strains of the WNV and are preferred for use in the present invention:

| | |
|---|---|
| CTCTTCCTCTCCGT (position 2443): 25 matches | (SEQ ID NO: 2) |
| TTCTTCCTCCTCATGC (position 6670): 30 matches | (SEQ ID NO: 3) |

The following information references the gene bank source for the sequence used.

*West Nile Virus AF404756.1

LOCUS AF404756 11029 bp ss-RNA linear VRL 23-JUL.-2002

DEFINITION West Nile virus isolate WN NY 2000-crow3356, complete genome.

ACCESSION AF404756

VERSION AF404756.1 GI:21929238

SOURCE West Nile virus.

ORGANISM West Nile virus

Viruses; ssRNA positive-strand viruses, no DNA stage; Flaviviridae;

Flavivirus; Japanese encephalitis virus group.

REFERENCE 1 (bases 1 to 11029)

AUTHORS Lanciotti, R. S., Ebel, G. D., Deubel, V., Kerst, A. J., Murri, S., Meyer, R., Bowen, M., McKinney, N., Morrill, W. E., Crabtree, M. B., Kramer, L. D. and Roehrig, J. T.

TITLE Complete genome sequences and phylogenetic analysis of West Nile virus strains isolated from the United States, Europe, and the Middle East

JOURNAL Virology 298 (1), 96-105 (2002)

MEDLINE 22089180

PUBMED 12093177

REFERENCE 2 (bases 1 to 11029)

AUTHORS Ebel, G. D., Kerst, A. J. and Lanciotti, R. S.

TITLE Direct Submission

JOURNAL Submitted (02-AUG.-2001) Division of Vector-Borne Infectious Diseases, Centers for Disease Control & Prevention, Rampart Road, Fort Collins, Colo. 80521, USA

One embodiment of the invention includes a method for testing a sample for the presence of at least one strain of West Nile Virus comprising providing a sample, the sample optionally containing West Nile Virus RNA, and exposing the sample to an oligomer having a targeting base sequence substantially complementary to at least seven consecutive bases in a WNV target sequence, then allowing the oligomer to hybridize with the WNV RNA to form a hybrid, then detecting the hybrid, thereby detecting the presence of at least one strain of the West Nile Virus. Another embodiment includes the method of testing a sample for the presence for a strain of WNV as described above, where the WNV target sequence includes any of the sequences set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ and SEQ ID NO: 9.

Another embodiment of the invention includes a method for testing a sample for the presence of a strain of WNV, comprising providing a sample, the sample optionally containing WNV RNA, and exposing the sample to an oligomer having a targeting base sequence that is substantially complementary to at least seven consecutive bases in a WNV target sequence, allowing the oligomer to hybridize with the WNV RNA to form a hybrid, and detecting the hybrid. The WNV target sequence includes a WNV RNA sequence comprising about seven to about 21 pyrimidine nucleotides with the WNV RNA sequence including no more than three purines within the pyrimidine sequence. Another embodiment of the invention includes using in the methods described above an oligomer that includes a parallel-stranded hairpin. The parallel-stranded hairpin may also include at least one 8-aminopurine.

A further embodiment of the invention includes a method for detecting the presence of a t least one strain of WNV in a sample by providing a sample, the sample optionally containing at least one strain of WNV RNA and exposing the sample to an oligomer having a targeting base sequence that is substantially complementary to at least about seven consecutive bases in a WNV target sequence, allowing the oligomer to hybridize with the WNV RNA to form a hybrid, detecting the hybrid, and thereby detecting the presence of at least one strain of WNV in the sample. The WNV target sequence is a sequence homologous to any of the sequences set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9.

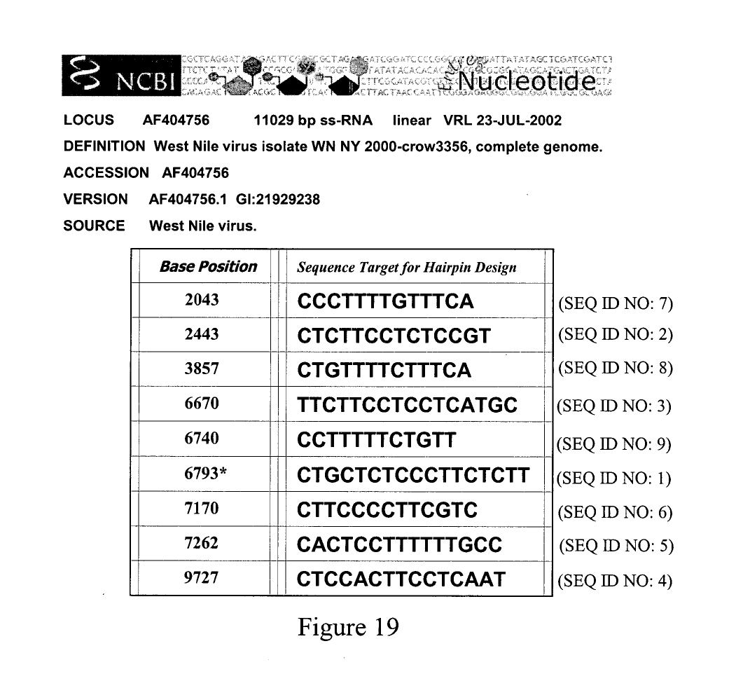

Sequences of the invention can be used to capture and/or report targeted RNA in a method for testing a sample for the presence of at least one strain of the West Nile Virus. The sequences are those found on FIG. 19 or any other polypyrimidine sequence of 7 to 21 nucleotides specific to the WNV genome that contain up to three purines within the pyrimidine sequence. Sequences that can be used as targets in a method for the treatment of patients infected with the WNC, using PSH probes or any other form of gene therapy. The sequences are those found on FIG. 19 or any other polypyrimidine sequence of 7 to 21 nucleotides specific to the WNV genome that contain up to three purines within the pyrimidine sequence.

Another embodiment of the invention includes a nucleic acid probe for detecting a target sequence of West Nile Virus RNA optionally present in a sample, the nucleic acid probe comprising a targeting base sequence that is substantially complementary to at least about seven consecutive bases in a West Nile Virus target sequence. A mixture of these nucleic acid probes may also be used.

Another embodiment of the invention describes the nucleic acid probe described above, where the nucleic acid probe includes a parallel-stranded hairpin. The parallel-stranded hairpin may further include at least one 8-aminopurine. In another embodiment of the invention, the nucleic acid probe is capable of hybridizing with a base sequence substantially complementary to at least about seven consecutive base sequences in at least one WNV target sequence. In a further embodiment of the invention this WNV target sequence is selected from the group including SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9.

Another embodiment of the invention includes a method for treating patients having the WNV, comprising administering to that patient a therapeutic amount of a composition capable of binding the RNA of the WNV, wherein the composition comprises an oligonucleotide having a base sequence that is substantially complementary to at least about seven consecutive bases in a West Nile Virus target sequence.

Another embodiment of the invention includes a method for capturing RNA of the WNV, including the steps of providing at least one oligomer probe. The oligomer probe includes a targeting base sequence substantially complementary to at least about seven consecutive bases in a WNV target sequence. The oligomer probe may further include an attached magnetic bead. The method further includes providing a sample, optionally containing RNA of the WNV, and combining the oligomer probe with the sample, forming at least one probe-RNA hybrid, and finally separating the probe-RNA hybrid from the sample by applying the magnetic field to the probe-sample mixture. This method may be performed where the WNV target sequence includes on one or more of the sequences set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9. The oligomer probe used in the method may include a parallel-stranded hairpin. This parallel-stranded hairpin may include at least one 8-aminopurine.

The invention further includes a method for inhibiting reproduction of the West Nile Virus comprising contacting the RNA of the West Nile virus with a composition capable of binding the RNA of the West Nile Virus, wherein the composition comprises an oligonucleotide having a base sequence that is substantially complementary to at least about seven consecutive bases in a West Nile Virus target sequence.

The invention provides a method of capturing the ribo-nucleic acid (RNA) specific to the West Nile Virus; specifically it relates to the target sequences within the WNV genome that can be used to capture the specific RNA material for identification and/or provide target regions for the use of specific probes that inhibit the reproduction of the WNV in vivo, in the form of a gene therapy drug or inhibit the reproduction of the WNV in vitro such as for example but not limited to a patient's blood products, tissues, organs, excretory products, and/or waste. A patient is any member of the animal kingdom, including but not limited to humans, dogs, cats, horses, birds, and pigs.

Whereas, particular embodiments of this invention have been described for purposes of illustration, it will be evident to those persons skilled in the art that numerous variations of the details of the present invention may be made without departing from the invention as defined in the appended claims. Furthermore, it will be evident to those persons skilled in the art that the claims of the invention are not meant to be bound to any particular theory.

What is claimed is:

1. A method for testing a sample for the presence of at least one strain of West Nile Virus, comprising providing a sample, said sample optionally containing West Nile Virus RNA, and exposing said sample to an oligomer comprising a targeting base sequence that is substantially complementary to at least seven consecutive bases in a West Nile Virus target sequence, allowing said oligomer to hybridize with said West Nile Virus RNA to form a hybrid, detecting said hybrid, and thereby detecting for the presence of at least one strain of West Nile Virus.

2. The method of claim 1, including wherein said oligomer comprises a parallel-stranded hairpin.

3. The method of claim 2, including wherein said parallel-stranded hairpin comprises at least one 8-aminopurine.

4. The method of claim 1, including wherein said West Nile Virus target sequence comprises the sequence set forth in SEQ ID NO: 1.

5. The method of claim 4, including wherein said oligomer comprises a parallel-stranded hairpin.

6. The method of claim 5, including wherein said parallel-stranded hairpin comprises at least one 8-aminopurine.

7. The method of claim 1, including wherein said West Nile Virus target sequence comprises the sequence set forth in SEQ ID NO: 2.

8. The method of claim 7, including wherein said oligomer comprises a parallel-stranded hairpin.

9. The method of claim 8, including wherein said parallel-stranded hairpin comprises at least one 8-aminopurine.

10. The method of claim 1, including wherein said West Nile Virus target sequence comprises the sequence set forth in SEQ ID NO: 3.

11. The method of claim 10, including wherein said oligomer comprises a parallel-stranded hairpin.

12. The method of claim 1 1, including wherein said parallel-stranded hairpin comprises at least one 8-aminopurine.

13. The method of claim 1, including wherein said West Nile Virus target sequence comprises the sequence set forth in SEQ ID NO: 4.

14. The method of claim 13, including wherein said oligomer comprises a parallel-stranded hairpin.

15. The method of claim 14, including wherein said parallel-stranded hairpin comprises at least one 8-aminopurine.

16. The method of claim 1, including wherein said West Nile Virus target sequence comprises the sequence set forth in SEQ ID NO: 5.

17. The method of claim 16, including wherein said oligomer comprises a parallel-stranded hairpin.

18. The method of claim 17, including wherein said parallel-stranded hairpin comprises at least one 8-aminopurine.

19. The method of claim 1, including wherein said West Nile Virus target sequence comprises the sequence set forth in SEQ ID NO: 6.

20. The method of claim 19, including wherein said oligomer comprises a parallel-stranded hairpin.

21. The method of claim 20, including wherein said parallel-stranded hairpin comprises at least one 8-aminopurine.

22. The method of claim 1, including wherein said West Nile Virus target sequence comprises the sequence set forth in SEQ ID NO: 7.

23. The method of claim 22, including wherein said oligomer comprises a parallel-stranded hairpin.

24. The method of claim 23, including wherein said parallel-stranded hairpin comprises at least one 8-aminopurine.

25. The method of claim 1, including wherein said West Nile Virus target sequence comprises the sequence set forth in SEQ ID NO: 8.

26. The method of claim 25, including wherein said oligomer comprises a parallel-stranded hairpin.

27. The method of claim 26, including wherein said parallel-stranded hairpin comprises at least one 8-aminopurine.

28. The method of claim 1, including wherein said West Nile Virus target sequence comprises the sequence set forth in SEQ ID NO: 9.

29. The method of claim 28, including wherein said oligomer comprises a parallel-stranded hairpin.

30. The method of claim 29, including wherein said parallel-stranded hairpin comprises at least one 8-aminopurine.

31. The method of claim 1, including wherein said West Nile Virus target sequence comprises a West Nile Virus RNA pyrimidine sequence comprising about seven to twenty-one nucleotides, said West Nile Virus RNA sequence comprising no more than three purines within the pyrimidine sequence.

32. The method of claim 31, including wherein said oligomer comprises a parallel-stranded hairpin.

33. The method of claim 32, including wherein said parallel-stranded hairpin comprises at least one 8-aminopurine.

34. The method of claim 1, including wherein said West Nile Virus Target Sequence is a sequence homologous to a sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9.

35. A nucleic acid probe for detecting a target sequence of West Nile Virus RNA optionally present in a sample, said nucleic acid probe comprising a targeting base sequence that is substantially complementary to at least about seven consecutive bases in a West Nile Virus target sequence.

36. The nucleic acid probe of claim 35, wherein said nucleic acid probe comprises a parallel-stranded hairpin.

37. The nucleic acid probe of claim 36, wherein said parallel-stranded hairpin comprises at least one 8-aminopurine.

38. A nucleic acid probe solution, comprising a mixture of nucleic acid probes of claim 35.

39. The nucleic acid probe of claim 35, wherein said West Nile Virus target sequence is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9.

40. A method for treating a patient having the West Nile Virus comprising administering to said patient a therapeutic amount of a composition capable of binding the RNA of the West Nile Virus, wherein said composition comprises an oligonucleotide having a base sequence that is substantially complementary to at least about seven consecutive bases in a West Nile Virus target sequence.

41. A method for capturing RNA of the West Nile Virus, comprising the steps of
a) providing at least one oligomer probe, said oligomer probe comprising a targeting base sequence that is substantially complementary to at least about seven consecutive bases in a West Nile Virus target sequence, said oligomer probe further comprising an attached magnetic bead;
b) providing a sample, said sample optionally containing RNA of the West Nile Virus;
c) combining said oligomer probe with said sample to form a mixture causing formation of at least one probe-RNA hybrid;
d) separating said probe-RNA hybrid from said sample by applying a magnetic field to said probe-sample mixture; and
e) capturing said RNA.

42. The method of claim 41, including wherein said West Nile Virus target sequence is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9.

43. The method of claim 41, including wherein said oligomer probe comprises a parallel-stranded hairpin.

44. The method of claim 41, including wherein said parallel-stranded hairpin comprises at least one 8-aminopurine.

45. A method for reporting RNA of the West Nile Virus, comprising the steps of
a) providing at least one oligomer probe, said oligomer probe comprising a targeting base sequence that is substantially complementary to at least about seven consecutive bases in a West Nile Virus target sequence, said oligomer probe further comprising an attached magnetic bead;
b) providing a sample, said sample optionally containing RNA of the West Nile Virus;
c) combining said oligomer probe with said sample to form a mixture causing formation of at least one probe-RNA hybrid;
d) separating said probe-RNA hybrid from said sample by applying a magnetic field to said probe-sample mixture; and
e) reporting said RNA.

46. A method for inhibiting reproduction of the West Nile Virus comprising contacting the RNA of the West Nile virus with a composition capable of binding the RNA of the West Nile Virus, wherein said composition comprises an oligonucleotide having a base sequence that is substantially complementary to at least about seven consecutive bases in a West Nile Virus target sequence.


SEQUENCE LISTING (25 sequences)

SEQ ID NO: 1 - DNA, 17 bp, West Nile Virus
  ref <308>: DATABASE ACCESSION NUMBER: AF404756
  ref <309>: DATABASE ENTRY DATE: 2002-07-23
  ref <313>: RELEVANT RESIDUES: (1)..(17)
ctgctctcccttctctt

SEQ ID NO: 2 - DNA, 14 bp, West Nile Virus
  ref <308>: DATABASE ACCESSION NUMBER: AF404756
  ref <309>: DATABASE ENTRY DATE: 2002-07-23
  ref <313>: RELEVANT RESIDUES: (1)..(14)
ctcttcctctccgt

SEQ ID NO: 3 - DNA, 16 bp, West Nile Virus
  ref <308>: DATABASE ACCESSION NUMBER: AF404756
  ref <309>: DATABASE ENTRY DATE: 2002-07-23
  ref <313>: RELEVANT RESIDUES: (1)..(16)
ttcttcctcctcatgc

SEQ ID NO: 4 - DNA, 15 bp, West Nile Virus
  ref <308>: DATABASE ACCESSION NUMBER: AF404756
  ref <309>: DATABASE ENTRY DATE: 2002-07-23
  ref <313>: RELEVANT RESIDUES: (1)..(15)
ctccacttcctcaat

SEQ ID NO: 5 - DNA, 15 bp, West Nile Virus
  ref <308>: DATABASE ACCESSION NUMBER: AF404756
  ref <309>: DATABASE ENTRY DATE: 2002-07-23
  ref <313>: RELEVANT RESIDUES: (1)..(15)
cactccttttttgcc

SEQ ID NO: 6 - DNA, 13 bp, West Nile Virus
  ref <308>: DATABASE ACCESSION NUMBER: AF404756
  ref <309>: DATABASE ENTRY DATE: 2002-07-23
  ref <313>: RELEVANT RESIDUES: (1)..(13)
cttccccttcgtc

SEQ ID NO: 7 - DNA, 13 bp, West Nile Virus
  ref <308>: DATABASE ACCESSION NUMBER: AF404756
  ref <309>: DATABASE ENTRY DATE: 2002-07-23
  ref <313>: RELEVANT RESIDUES: (1)..(13)
cccttttgtttca

SEQ ID NO: 8 - DNA, 13 bp, West Nile Virus
  ref <308>: DATABASE ACCESSION NUMBER: AF404756
  ref <309>: DATABASE ENTRY DATE: 2002-07-23
  ref <313>: RELEVANT RESIDUES: (1)..(13)
ctgttttctttca

SEQ ID NO: 9 - DNA, 12 bp, West Nile Virus
  ref <308>: DATABASE ACCESSION NUMBER: AF404756
  ref <309>: DATABASE ENTRY DATE: 2002-07-23
  ref <313>: RELEVANT RESIDUES: (1)..(12)
cctttttctgtt

SEQ ID NO: 10 - DNA, 13 bp, Artificial
  Hairpin Component
  misc_feature (4)..(4): n is 8-aminoguanine
  misc_feature (6)..(6): n is 8-aminoadenine
aaananggaggag

SEQ ID NO: 11 - DNA, 13 bp, Artificial
  Hairpin Component
tttcttcctcctc

SEQ ID NO: 12 - DNA, 11 bp, Artificial
  hairpin component
cttcctcctct

SEQ ID NO: 13 - DNA, 11 bp, Artificial
  hairpin component
gaaggaggaga

SEQ ID NO: 14 - DNA, 11 bp, Artificial
  hairpin component
tctcctccttc

SEQ ID NO: 15 - DNA, 11 bp, Artificial
  hairpin component
  misc_feature (6)..(6): n is 8-aminoadenine
  misc_feature (9)..(9): n is 8-aminoadenine
gaaggnggnga

SEQ ID NO: 16 - DNA, 11 bp, Artificial
  hairpin component
  misc_feature (5)..(5): n is 8-aminoguanine
  misc_feature (8)..(8): n is 8-aminoguanine
gaagnagnaga

SEQ ID NO: 17 - DNA, 11 bp, Artificial
  triplex target component
tctcctccttc

SEQ ID NO: 18 - DNA, 11 bp, Artificial
  triplex target component
tctcctgcttc

SEQ ID NO: 19 - DNA, 11 bp, Artificial
  hairpin component
  misc_feature (6)..(6): n is 8-aminoadenine
  misc_feature (9)..(9): n is 8-aminoadenine
gaagcnggnga

SEQ ID NO: 20 - DNA, 13 bp, Artificial Sequence
  hairpin component
  misc_feature (7)..(7): n is 8-aminoguanine
  misc_feature (10)..(10): n is 8-aminoguanine
gaagganganatt

SEQ ID NO: 21 - RNA, 13 nt, Artificial
  Hairpin Component
cuuccuccucuuu

SEQ ID NO: 22 - RNA, 11 nt, Artificial
  target for hairpin
ucuccuccuuc

SEQ ID NO: 23 - DNA, 13 bp, Artificial
  AR22 synthesis component
gaaggaggagatt

SEQ ID NO: 24 - DNA, 13 bp, Artificial
  AR22 synthesis component
cttcctcctcttt

SEQ ID NO: 25 - DNA, 11 bp, Artificial
  control hairpin component
tttttcccccc